(19) United States
(12) Patent Application Publication
(10) Pub. No.: US 2010/0241046 A1
(43) Pub. Date: Sep. 23, 2010

(54) APPARATUS, METHODS AND DEVICES FOR TREATMENT OF OCULAR DISORDERS

(75) Inventors: Leonard Pinchuk, Miami, FL (US); Jean-Marie A. Parel, Miami Shores, FL (US); Francisco Fantes, Key Biscayne, FL (US)

Correspondence Address: GORDON & JACOBSON, P.C., 60 LONG RIDGE ROAD, SUITE 407, STAMFORD, CT, 06902, US

(73) Assignee: INNFOCUS, LLC, Mami, FL (US)

(21) Appl. No.: 12/440,096

(22) PCT Filed: Sep. 6, 2007
(86) PCT No.: PCT/US07/77757

§ 371 Date: Jun. 4, 2010

Related U.S. Application Data
(60) Provisional application No. 60/824,632, filed on Sep. 6, 2006.
(60) Provisional application No. 60/825,595, filed on Sep. 14, 2006.

(51) Int. Cl. A61M 1/00

(52) U.S. Cl. 604/8

(57) ABSTRACT

Apparatus and methods for relieving and treating glaucoma and other ocular disorders are disclosed. The apparatus includes a thin flexible membrane and preferably a tubular member. In another aspect of the invention, a surgical device for repairing ocular tissue includes a flexible membrane of a polymeric material comprising polyisobutylene. In the preferred embodiment, the polymeric material of the membrane is porous.

### CROSS REFERENCED RELATED APPLICATIONS

This application claims the benefit of U.S. Provisional Application Ser. Nos. 60/824,632 filed Sep. 6, 2006 and 60/825,595 filed on Sep. 14, 2006 and is related to International Patent Appl. No. PCT/US07/77731, entitled “Porous Polymeric Material For Medical Applications,” filed concurrently herewith (Attorney Docket No. INN-025 PCT), all of which are herein incorporated by reference in their entireties.

### BACKGROUND OF THE INVENTION

1. Field of the Invention

This invention relates broadly to methods and apparatus for treating diseases and disorders of the eye such as glaucoma.

2. State of the Art

Glaucoma is a leading cause of blindness. It is the direct result of poor drainage flow of aqueous humor from the anterior portion of the eye. When poor drainage occurs, intraocular pressure in the eye increases which in turn causes damage to the optic nerve through loss of retinal ganglion cells. Glaucoma onsets in a gradual manner whereby the victim rarely recognizes the increasing loss of peripheral vision as the disease progresses.

Glaucoma is generally categorized into one of two types. In open-angle glaucoma, the impaired outflow is caused by abnormalities of the drainage system of the anterior chamber. In closed-angle glaucoma, the impaired outflow is caused by impaired access of aqueous humor to the drainage system. If the pressure within the eye remains sufficiently high for a long enough period of time, total vision loss occurs. Glaucoma is the number one cause of preventable blindness.

Proper flow of aqueous humor within the human eye is crucial to preventing glaucoma. Aqueous humor is a clear fluid contained in the eye formed by a ciliary body adjacent the posterior chamber of the eye. The fluid is made at a nearly constant rate before passing the lens and iris of the eye and entering the anterior chamber of the eye. It is in the anterior chamber that the aqueous humor drains out in one of two ways. Approximately ten percent of aqueous humor drainage occurs by the percolation of aqueous humor between muscle fibers of the ciliary body in the “uveoscleral” route. Aqueous humor primarily flows out through the “canalicular” route via the trabecular meshwork and Schlemm's canal.

In a properly functioning eye, aqueous humor production equals aqueous outflow and intraocular pressure remains fairly constant (typically in the 15 to 21 mmHg range). In glaucoma patients however, there is abnormal resistance to aqueous outflow which in turn results in an increase in intraocular pressure. With the increased resistance, the aqueous humor fluid pressure builds because it cannot exit properly. As the fluid pressure builds, the intraocular pressure within the eye increases. The increased intraocular pressure compresses the axons in the optic nerve and also may compromise the vascular supply to the optic nerve. The optic nerve carries vision from the eye to the brain. Some optic nerves are more susceptible to increases in intraocular pressure than others.

Medication is often a first option in the treatment of glaucoma. Administered either topically or orally, these medications work to either reduce aqueous production or they act to increase outflow. However, currently available medications have many serious side effects including congestive heart failure, respiratory distress, hypertension, depression, renal stones, aplastic anemia, and sexual dysfunction. Some medication treatments for glaucoma may be fatal. Furthermore, administration of glaucoma medication is a major problem with estimates of over half of glaucoma patients improperly following correct dosing schedules.

Laser trabeculoplasty is performed as an alternative to medication. This process applies thermal energy from a laser to a number of noncontiguous spots in the trabecular meshwork. It is believed that the laser energy stimulates the metabolism of the trabecular cells in some way, and changes the cellular material in the trabecular meshwork. In a large percent of patients, aqueous outflow is enhanced and intraocular pressure decreases. However, the effect often is transient and a significant percentage of patients develop an elevated eye pressure within the years that follow the treatment. Laser trabeculoplasty treatment is typically not repeatable. In addition, laser trabeculoplasty is not an effective treatment for primary open angle glaucoma in patients less than fifty years of age, nor is it effective for angle closure glaucoma and many secondary glaucomas.

If laser trabeculoplasty does not reduce the pressure sufficiently, then incisional surgery (typically referred to as filtering surgery) is performed. With incisional surgery, a hole is made in the sclera adjacent the angle region. This hole allows the aqueous fluid to leave the eye through an alternate route.

The most commonly performed incisional procedure is a trabeculectomy. In a trabeculectomy, a posterior incision is made in the conjunctiva, which is the transparent tissue that covers the sclera. The conjunctiva is rolled forward, exposing the sclera at the limbus, which marks the junction between the sclera and the cornea. A partial scleral flap is made and dissected into the cornea. The anterior chamber is entered beneath the scleral flap, and a section of deep sclera and trabecular meshwork is excised. The scleral flap is loosely sewn back into place. The conjunctiva incision is tightly closed. Post-operatively, the aqueous fluid passes through the hole, beneath the scleral flap and collects in a bleb formed beneath the conjunctiva. The fluid then is either absorbed through blood vessels in the conjunctiva or traverses across the conjunctiva into the tear film. Trabeculectomy surgery of this nature is extremely difficult and only a small fraction of ophthalmologists perform this procedure. In addition, it is very time consuming and physicians are not reimbursed for the time it takes to perform the surgery and it is therefore rarely performed.

The final alternative to lower intraocular pressure is a surgical procedure that implants a device that shunts aqueous humor. An example of such a device (as shown in U.S. Pat. No. 6,050,970 to Baerveldt) is a drainage tube that is attached at one end to a plastic plate. The drainage tube is a flow tube between 1.0 and 3.0 French (and preferably with an inner diameter of 0.3 mm and an outer diameter of 0.6 mm). An incision is made in the conjunctiva exposing the sclera. Often times the muscles that enable rotation of the eye are partially dissected from the sclera to allow placement of the plastic plate. The plastic plate is sewn to the surface of the eye posteriorly, usually over the equator. A full thickness hole is made into the eye at the limbus, usually with a needle. The tube is inserted into the eye through this hole. The external portion of the tube is covered with cadaver sclera, cornea, or other tissue. The conjunctiva is replaced and the incision is closed tightly. With this shunt device, aqueous drains from the interior of the eye through the silicone tube to the dissection plane where the plastic plate is placed. As the dissection plane fills with aqueous humor it forms a bleb, which is a thin layer of connective tissue that encapsulates the plate and tube. Aqueous drains out of the bleb and to the surface of the eye into the tear ducts or to the venous circulation within the deeper orbital tissues. The plate typically has a large surface area in order to wick and disperse fluid, which facilitates absorption of fluid in the surrounding tissue. These disks are generally made of silicone rubber, which serves to inhibit tissue adhesion as the plate becomes encapsulated by the connective tissue of the bleb. The disks can be as large as 10 mm in diameter and are irritating to some patients. Further the tissue that encapsulates these plates can be thick and restrict rotation of the eye resulting in diplopia or double vision.

Other implant devices are shown in U.S. Pat. No. 6,468,283 to Richter et al. and U.S. Pat. No. 6,626,858 to Lynch et al. The Richter implant device is a tubular structure that shunts aqueous humor from the anterior chamber to a space between the conjunctiva and the sclera. The Lynch implant device is a tubular structure that shunts aqueous humor from the anterior chamber through the trabecular meshwork and into Schlemm's canal. These implant devices are described as being formed from silicone, Teflon, polypropylene, stainless steel, etc. These implant devices also typically require precise placement away from the angle and the iris in order to prevent interference with the iris and/or to avoid occlusion of the drainage lumen by ocular tissue (for example, the fibrous tissue of the iris and/or the sclera that may plug the drainage lumen). In addition, such implant devices typically include a unidirectional valve to minimize hypotony (low intraocular pressure) in the anterior chamber of the eye. However, the desired flow control provided by such valves is difficult to maintain and are prone to failure. Lastly, these shunt devices are relatively stiff and have been shown to erode through the ocular tissue wall adjacent thereto over time.

Thus, there remains a need in the art to provide an implant device for the treatment of glaucoma that is realized from a biocompatible material which will not encapsulate in the eye and that enables control over intraocular pressure without the need for large surface area plates and has a softness that will not irritate the eye and surrounding tissue structures.

### SUMMARY OF THE INVENTION

It is therefore an object of the present invention to provide a treatment apparatus that facilitates the drainage of aqueous humor from the anterior portion of an eye that does not interfere with the normal operation of the eye.

It is still another object of the invention to provide an apparatus for use in the eye that is sufficiently thin and soft and will not irritate the eye when implanted.

It is yet another object of the invention to provide an apparatus for use in relieving intraocular pressure within the eye that is biocompatible and will not encapsulate.

In accord with these objects, which will be discussed in detail below, an apparatus and method for draining aqueous humor is provided for relieving pressure within the eye. The apparatus includes a tubular member and a flexible membrane. The tubular member drains aqueous humor from the anterior chamber of the eye to the flexible member. An end portion of the tubular member is inserted directly into the anterior chamber of the eye. In use, the flexible membrane forms a bleb which acts as a reservoir for diffusion of aqueous humor into the ocular environment. The membrane is formed of a polymeric material that prevents the bleb from healing closed and enables the bleb to be thin. The flexible member and thus the bleb formed thereby can be positioned under the conjunctiva and under the Tenons preferably between the conjunctiva/Tenon and the sclera. Isolating these tissues provides a large surface equivalent to twice the membrane's planar surface so that aqueous fluids, if present, can dissipate by wicking along the membrane surfaces and absorb via the episclera into the choroidal space and via Tenon's connective tissues into the conjunctival space. Both spaces are maintained by the body at a low interstitial pressure.

The membrane can also be implanted alone or in conjunction with a trabeculectomy. In this embodiment of the invention, the membrane functions to prevent natural reattachment of the scleral tissue. It can also be implanted into the ocular environment for other purposes.

The tubular member and the membrane of the present invention are preferably both made from a block copolymer of polystyrene and polyisobutylene material (herein after referred to as SIBS). When used to realize the membrane, the SIBS copolymer enables the membrane and tissues surrounding the bleb to be thin and therefore requires less surface area than other biocompatible materials. Furthermore, SIBS can be made as soft as surrounding skin tissues by varying the relative amounts of polystyrene and polyisobutylene contained in the copolymer and will not encapsulate when implanted within the human body. This placement results in the formation of a permeable sheath of tissue through which aqueous humor can penetrate.

The membrane can be realized from a non-porous polymeric structure or porous polymeric structure. If non-porous, the membrane does not allow aqueous humor to flow through the material structure. If porous, the membrane allows aqueous humor to freely flow through the material structure. SIBS can be made with varying degrees of porosity typically ranging from 30% to 70%.

Additional objects and advantages of the invention will become apparent to those skilled in the art upon reference to the detailed description taken in conjunction with the provided figures.

### BRIEF DESCRIPTION OF THE DRAWINGS

FIG. 1 is a pictorial illustration of a prior art aqueous humor drainage tube implanted in the ocular environment;

FIGS. 2-4 are schematic diagrams of embodiments of a membrane in accordance with the present invention;

FIG. 5 is a schematic diagram that illustrates the use of the aqueous humor drainage tube of FIG. 1 with the membrane of FIG. 4 in accordance with the present invention;

FIG. 6 is a schematic diagram that illustrates a mechanism for attaching the aqueous humor drainage tube of FIG. 1 with the membrane of FIG. 4 in accordance with the present invention;

FIG. 7 is a schematic diagram that illustrates the use of the aqueous humor drainage tube of FIG. 1 with the membrane in accordance with the present invention; and

FIGS. 8A-8D are schematic diagrams that illustrate mechanisms for integrally attaching the aqueous humor drainage tube of FIG. 1 with the membrane in accordance with the present invention.

FIG. 9 is a schematic diagram of a second embodiment of the membrane.

### DETAILED DESCRIPTION OF THE PREFERRED EMBODIMENTS

As used herein, the term “porous” refers to a material or structure that has a plurality of holes, perforations, openings, or void spaces (collectively, “pores”).

As used herein, the term “microporous” refers to a material or structure that has a plurality of pores with an average pore size of less than 200 microns. For purposes of this application, pore size shall mean the largest dimension of the pore.

As used herein, the term “porosity” refers to the ratio of non-solid volume to the total volume of a porous material.

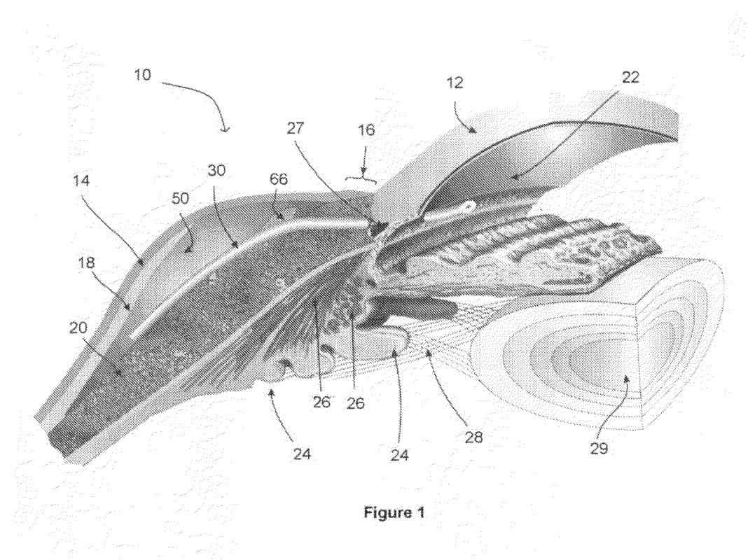

Turning now to FIG. 1, a schematic view of a portion of an eye 10 is shown. A cornea 12 of the eye 10 is joined to a conjunctiva 14 at a limbus 16. The conjunctiva 14 is a protective layer surrounding a sclera 20 of the eye 10. The sclera 20 is a more rigid protective layer of tissue that surrounds the internal structures of the eye. Tenon's connective tissues 18 are shown just below the conjunctiva 14 above the sclera 20.

Aqueous humor is maintained in an anterior portion 22 of the eye 10. In a normally functioning eye, intraocular pressure is maintained at a fairly constant level as aqueous humor is produced by a ciliary body 24 and drained out from the anterior portion 22 via Schlemm's canal 27. Schlemm's canal 27 is a circular channel and delivers aqueous humor to the blood stream after collecting it from the anterior portion 22.

In addition to the producing aqueous humor, the ciliary body 24 has the important function of maintaining muscles for controlling the shape of a lens 29 in the eye. Ciliary muscles 26 located within the ciliary body 24 control zonule fibers 28. These zonule fibers 28 join the ciliary body 24 to the lens 29 and respond to movements by the ciliary muscles 26. Selective motion of the zonule fibers 28 in turn assist in controlling the shape of the lens 29 for proper reception and focusing of light.

FIG. 1 shows an aqueous humor drainage tube 30 as described in U.S. Application No. 60/741,514, filed on Dec. 1, 2005 herein incorporated by reference in its entirety. A proximal end of the tube 30 is inserted into an anterior chamber 22 of the eye 10, and a distal end is inserted into a flap 50. The flap 50 is formed between the conjunctiva 14, Tenon's connective tissues 18, and the sclera 20. When aqueous humor fluid drains from the anterior chamber 22 into the flap 50, a blister is formed which ophthalmologists refer to as a bleb.

Experimentation with the tube 30 of FIG. 1 in certain patients having severe glaucoma resulted in the bleb healing closed due to exuberant fibrotic contraction. This closure resulted in sealing of the fluid reservoir created shortly after surgery undermining the value of the tube 30. More simply, the bleb disappeared and fluid stopped draining from the anterior chamber.

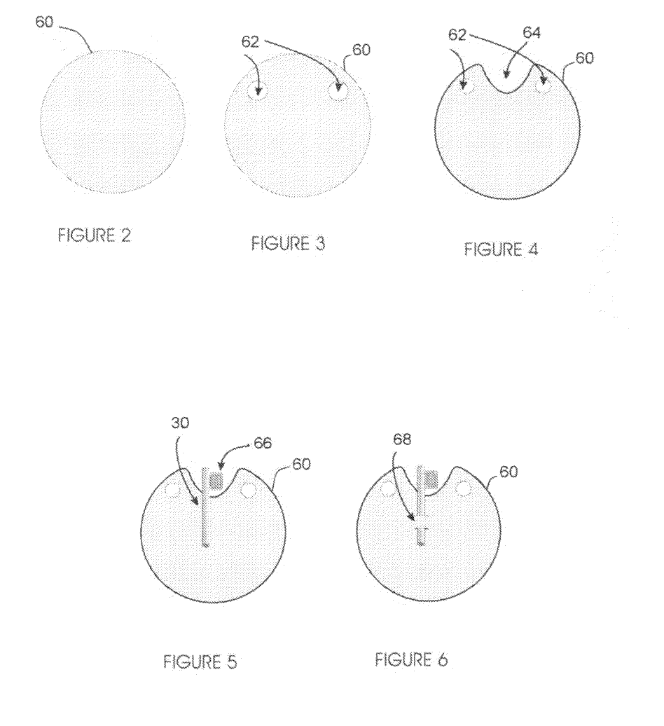

Turning now to FIGS. 2-7, an aqueous humor drainage system in accordance with the present invention includes a section of the drainage tube of FIG. 1 used in conjunction with a thin flexible membrane 60. In use, the membrane 60 forms a bleb which acts as a reservoir for the diffusion of aqueous humor into the ocular environment. The membrane 60, shown in FIG. 2 in its most simplistic form as a circular disk, is made from biocompatible polymers such as polyisobutylene or SIBS with a Shore hardness between Shore 10 A and 90 A, most preferably Shore 45 A. The hardness of SIBS is controlled by varying the relative amounts of polyisobutylene and polystyrene in the block copolymer. As the weight fraction of polyisobutylene in the SIBS copolymer approaches 1 (that is, no styrene in the copolymer), the copolymer approaches Shore hardness values of 10 A and similarly as the weight fraction of polystyrene is increased, the Shore hardness of the SIBS increases and can readily approach 80 A or more.

The membrane is preferably between 0.020 millimeters (mm) and 0.6 mm thick and most preferably between 0.04 mm and 0.5 mm thick. These ranges of thickness are more compatible with the surface of the episclera and therefore the membrane 60 becomes less traumatic to the eye with the least interference to normal rotation of the operated eye. Abnormal rotation of the operated eye can cause unwanted diplopia wherein the eyes of a patient do not rotate congruously with respect to each other. It is also important that the membrane 60 is not too thin because it becomes more difficult to maneuver surgically and it may fold onto itself.

The membrane 60 can be made as a porous or non-porous structure. However, the use of a porous polymer such as SIBS or polyisobutylene offers significant additional benefits over other materials when used in the eye for both the tubular member and the membrane. When a tube derived from SIBS is implanted in the human body, the SIBS material prevents encapsulation thereby inhibiting the body's natural healing mechanisms from closing the entrance to the tube opening. Placement of the SIBS tube also results in formation of a very thin permeable sheath of tissue through which aqueous humor can readily diffuse. Also, when SIBS is used in the membrane to form a bleb, the bleb does not heal closed allowing continuous drainage of aqueous humor similar to the flow of a normally functioning eye. The SIBS membrane isolates the episclera from the conjunctiva 14 and Tenon's connective tissues 18 thereby preventing their natural reattachment. The isolation of these tissues together with the permeability and non-encapsulating characteristics of the membrane provides a large surface equivalent to about twice the membrane's planar surface area so that aqueous fluids can dissipate by wicking along the membrane surfaces and absorbing via the episclera into the choroidal space and via Tenon's connective tissues into the conjunctival space and via the conjunctival space into the tear ducts. These spaces are maintained by the body at a low interstitial pressure. Such dissipation provides improved outflow of aqueous fluids over the surfaces of the membrane and allows the membrane to be small as compared to the prior art glaucoma plates. In the preferred embodiment, the membrane 60 covers a surface area in a range between 40 mm² and 150 mm² and more preferably in a range between 50 mm² and 150 mm².

In addition, SIBS is highly biocompatible and less subject to biodegradation when compared to many other implanted materials. A non-porous SIBS membrane does not allow for aqueous humor to flow through the membrane. A porous SIBS membrane provides numerous fluid pathways that allow for aqueous humor to flow through the membrane and circulate within the bleb. In addition, porosity makes the SIBS material foam-like and thereby improves the membrane's flexibility and physical compatibility with surrounding tissue structures, thus making the membrane atraumatic.

SIBS can be made porous using a number of processes. In a first example, SIBS is made porous by a phase inversion technique, where SIBS is dissolved in a good solvent such as hexane and then poured into a container to shape the membrane. However, instead of flashing off the good solvent, which would provide a cast non-porous membrane, a poor solvent, such as isopropyl alcohol is added to the solution such that the good solvent migrates into the poor solvent and the SIBS precipitates out. As solvent migrates out from the precipitant, the precipitant is left with interconnected pores. When dried, the precipitated SIBS becomes a porous membrane structure. Pore size is controlled by applying the solution of SIBS and good solvent to a porous mandril. In this way the polymer can be made microporous when a microporous mandril is used. This first example is described in U.S. Patent Application Publication US2005/0055075 to Pinchuk, herein incorporated by reference in its entirety.

In a second example, a SIBS copolymer can be melted with a second polymer. The second polymer acts as a sacrificial polymer component when dissolved by a solvent. Upon equilibration with a solvent capable of dissolving the sacrificial polymer component, the soluble polymer elutes out from the SIBS copolymer leaving pores behind where the sacrificial component previously resided. The pore size is controlled by controlling the ratio of the SIBS polymer to the sacrificial polymer. When more SIBS polymer is used, the pore size becomes effectively smaller due to more tortuosity through the membrane. In addition, the molecular weight of the sacrificial polymer can be used to control pore size; the larger the molecular weight, the larger the pore size. This second example is described in detail in International Patent App. No. PCT/US07/77731 entitled “Porous Polymeric Material For Medical Applications,” filed concurrently herewith (Attorney docket number INN-025), which is incorporated by referenced above. These examples present merely two ways in which pores may be formed within the SIBS material. Other processes for creating porosity can be used as envisioned by one of ordinary skill in the chemical arts.

As seen in FIG. 3, the membrane 60 can have one or more holes 62. The holes 62 enable the surgeon to suture the membrane 60 to the sclera 20. Although placement of the holes 62 are shown to be approximately 120 degrees apart from each other relative to the center of the membrane 60, the holes 62 can be formed in the membrane at any desirable location to aid suturing. By fixing the membrane 60 to the sclera 20, migration of the membrane 60 within the flap is prevented. Alternatively, the membrane 60 may be affixed to the sclera using a biocompatible glue, clips or other attachment means as envisioned by one of ordinary skill in the art. Additional holes can be added or removed from the membrane 60 as needed to facilitate tissue ingrowth into the apparatus. Such tissue ingrowth may help to secure the apparatus in place and also keep it from inadvertently folding.

As seen in FIG. 4, the membrane 60 is shown having an indentation 64. Continuing to FIG. 5, the tube 30 can be positioned across the body of the membrane 60. In this configuration, the tube 30 can have a fixation member 66 that projects through the space created by the indentation 64. The indentation 64 allows the fixation member 66 to maintain a lower profile than if the fixation member 66 or the tube 20 merely rested against the membrane 60. Without the indentation 64, the fixation member 66 may protrude too far upward and slowly erode through the conjunctiva 14. The fixation member 66 as shown in FIGS. 5-6 is a fin but may also take form of a tab or equivalent structure to secure the position of the tube 30 against the conjunctiva 14.

As seen in FIG. 6, membrane 60 may include a means for joining the tube 30 to the membrane 60. Here, the membrane 60 includes a band 68 cut in the membrane 60. The band 68 allows for insertion of the tube 30 through the band 68 and serves to hold the tube 30 in place relative to the membrane 60.

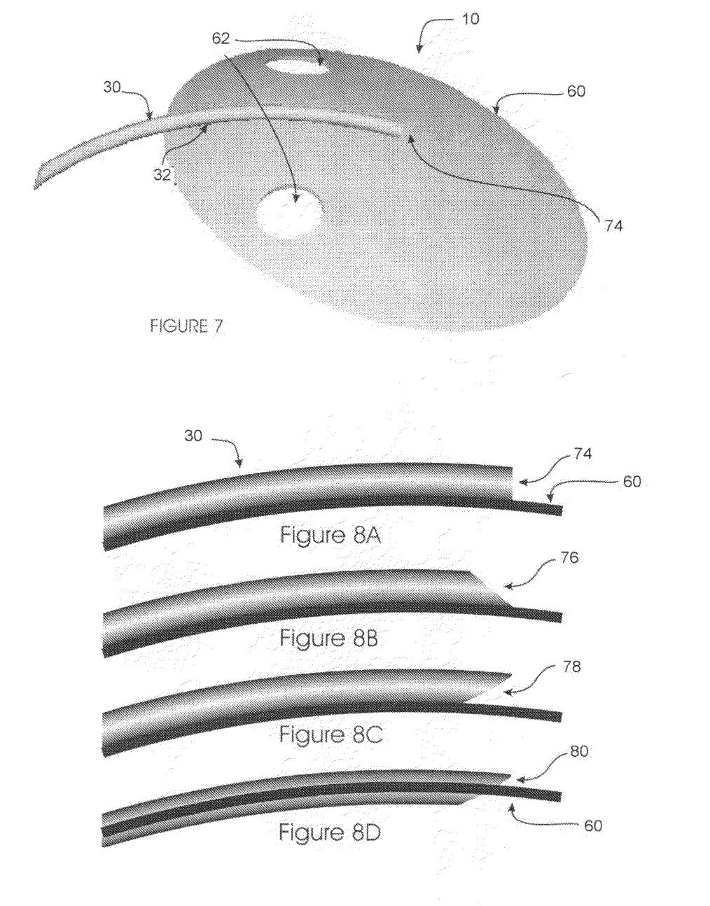

Referring to FIG. 7, alternative means for joining the two components of the apparatus can be envisioned by one of ordinary skill in the art such as the use of additional holes, multiple bands, or slots to name a few. The tube 30 may also be attached to the membrane 60 using solvent bonding, heat fusing, insert molding, and the like. Solvent bonding of the apparatus can be achieved by placing a drop of an appropriate solvent on the portion of the tube 30 that contacts the membrane 60. Appropriate solvents include non-polar solvents such as tetrahydrofuran, cyclopentane, toluene, cyclohexane, heptane, xylene, benzene, and the like. In order to prevent the solvent from dissolving through the tube or the membrane, it is preferred that up to 30% of SIBS be dissolved in the solvent to thicken it and thereby prevent it from dissolving the items to be bonded. Alternatively, the non-polar solvent can be diluted with a poor polar solvent such as 2-propanol to decrease its solvent potency and thereby avoid dissolving the two structures to be adhered together. Heat bonding of the apparatus can be achieved by first placing a wire mandrel in the tube lumen to prevent the lumen of the tube from heat welding closed. The tube is placed in a fixture and the membrane 60 is placed over the tube 30 and one or two hot dies press the tube 30 against the membrane 60 to a certain depth to create the melt bond. Insert molding of the apparatus can be achieved by inserting the tube into an insert mold cavity sized to outline the shape of the membrane 60 where the membrane 60 is formed and bonded to the tube 30 at the same time. The polymer is injected into the insert mold cavity to form the membrane 60 bonded to the tube 30. Insert molding has certain advantages in that the contour between the tube 30 and the membrane 60 can be precisely controlled.

FIG. 7 shows the combination of the tube 30 and the membrane 60 in an assembled configuration. In the configuration shown, the membrane 60 has a convex shape (e.g., similar to a yarmulke) to better fit the surface contour of the eye 10. The tube 30 is integrally attached to the membrane 60 along a locus between a central portion 32 of the tube 30 and a first end 74 of the tube 30. However, in some embodiments it may be preferred to adhere the tube 30 to the membrane 60 to enable tilting of the membrane 60 to facilitate placement in the flap.

Although FIG. 7 shows the membrane 60 having a convex shape (e.g., similar to a yarmulke), those skilled in the art of opthalmology will appreciate that all the designs shown in this disclosure can have such a convex shape. The membrane 60 has a diameter that is preferably 5 mm to 12 mm (and most preferably 6 mm to 8 mm). Although the figures provided depict the tube 30 resting on top of the membrane 60, the tube can also rest below the membrane 60 on the episcleral side. In another alternative, two membranes can be used with the tube located between them or the tube can be molded within the thickness of the membrane.

Turning now to FIGS. 8A-8D, various configurations of a first end 74 of tube 30 are shown. FIG. 8A shows the tube 30 resting on the surface of membrane 60 with the first end 74 cut at a 90° angle. FIG. 8B shows a first end 76 having an acute angle relative to the axis of the tube 30. FIG. 8C shows a first end 78 having an obtuse angle relative to the axis of the tube 30. FIG. 8D shows the tube 30 with a first end 80 having an obtuse angle melded partially into the membrane 60. The preferable angulation is that shown in FIG. 8C, because the overhang of the obtuse angle prevents tissue from clogging the exit of the tube. In FIG. 8D, the first end 80 of tube 30 is obtuse both below and above the membrane 60. A small hole may be placed near the first ends 74, 76, 78, 80 to facilitate communication across the membrane.

Also within the scope of this application but not shown is any of the configurations of FIGS. 8A-8D with the tube being comprised of a porous wall. The porous wall preferably has pores sized between 0.1 and 100 micrometers to enable fluid from the anterior chamber to seep and percolate in the episcleral, sub-Tenonian, and sub-conjunctival spaces. Also not shown is an embodiment that can be added to those structures in FIG. 8 where a thin membrane is placed over the tube to further prevent tissue from growing into the tube orifice.

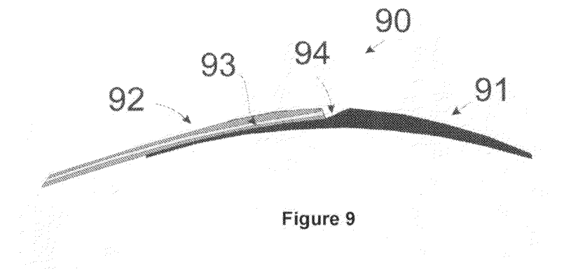

Turning now to FIG. 9, a second embodiment of the invention is shown as device 90 having a membrane 91 integrated with a tube 92. In this embodiment, the membrane 91 has a stepped interface 94 in its central portion. At this stepped interface 94, the thickness of the membrane transitions from a larger thickness to a smaller thickness that extends to the perimeter of the membrane 91. The tube 92 is shown in cross-section having a lumen 93 through which aqueous humor fluid is drained. The lumen of the tube has a diameter ranging from 0.05 mm to 0.20 mm and most preferably ranging from 0.70 mm to 0.15 mm. The tube 92 is disposed atop the small thickness part of the membrane 91 where it exits adjacent the stepped interface 94. In this manner, fluid draining through the tube 92 exits into a trench formed by the stepped interface 94 and wicks along the surface of the membrane 91 dissipating into adjacent conjunctiva/Tenon tissue or scleral tissue. Similar to the earlier described embodiment, the membrane 91 may be composed of a solid or porous material and is preferably made sufficiently thin so that it does not interfere with the normal operation of the eye. The thickness of the membrane 91 can be from a knife edge at its periphery to 0.6 mm thick at the thick part of the stepped interface 94, or possibly from 0.02 mm at the periphery to 0.5 mm at the thick part of the stepped interface 94. The advantage of the increased thickness at the stepped interface 94 is that it prevents the membrane 91 from inadvertently folding upon itself.

In the embodiments described above, the membrane and the tube can be implanted alone or in conjunction with a trabeculectomy. In this embodiment of the invention, the membrane functions to isolate the episclera from conjunctiva and Tenon's connective tissue, thereby preventing their natural reattachment. Isolating these tissues provides a large surface area equivalent to twice the membrane's planar surface so that aqueous fluids, if present, can dissipate by wicking along the membrane surfaces and absorb via the episclera into the choroidal space and via Tenon's into the conjunctival space, both spaces being maintained by the body at a low interstitial pressure. As the trabeculectomy is normally at most 4 mm×4 mm square, the polymeric membrane must be made smaller to fit into the trabeculectomy well. Membrane diameters can range from 1 mm to 4 mm with 2 mm being preferred. Further the membrane may be round or square with surface areas ranging form 1.5 mm² to 25 mm².

The polymeric membrane and the tube as described above can have one or more therapeutic agents loaded therein that elute out from the polymer material of the membrane to aid in preventing the bleb from healing. Exemplary therapeutic agents include: (1) antiproliferatives like paclitaxel, rapamycin, and the like; (2) antimetabolites such as mitomycin C, 5-fluorouracil and the like; (3) fibrolytic agents such as urokinase, streptokinase, TPa and the like; and (4) anticoagulants like heparin, analogues of heparin, adrenalin, epinephrine, aspirin, and the like, or cocktails of the above. A description of other drugs can be found in U.S. Pat. No. 6,545,097, herein incorporated by reference in its entirety. The drug(s) can be placed on either side or on both sides of the membrane or the entire membrane can be impregnated with one or more drug(s). The elution of the drug(s) can be immediate or over a period of weeks to months. In addition, the membrane can be coated with a blocking agent, such as glycerin, lecithin, bis-stearamide, polytetrafluoroethylene, polystyrene, silicone oil, and the like to prevent adhesions, both for handling purposes and to prevent tissue adhesion.

Drugs that are particularly useful in the treatment of eye disorders can be loaded into the membrane. As an example, for the treatment of macular degeneration (AMD), the membrane 60 can be loaded with a number of therapeutic agents, including: Paclitaxel, Macugen, Visudyne, Lucentis (rhuFab V2 AMD), Combretastatin A4 Prodrug, Squalamine, SnET2, H8, VEGF Trap, Cand5, LS11 (Taporfin Sodium), AdPEDF, RetinoStat, Integrin, Panzem, Retaane, Anecortave Acetate, VEGFR-1 mRNA, ARGENT cell-signalling technology, Angiotensin II Inhibitor, Accutane for Blindness, Macugen (PEGylated aptamer), PTAMD, Optrin, AK-1003, NX 1838, Antagonists of avb3 and 5, Neovastat, Eos 200-F and any other VEGF inhibitor.

If desired, a therapeutic agent of interest can be loaded at the same time as the polymer from which the membrane and tube are realized, for example, by adding the drug to a polymer melt during thermoplastic processing or by adding it to a polymer solution during solvent-based processing. Alternatively, a therapeutic agent can be loaded after formation of the membrane, tube, or portions thereof. As an example of these embodiments, the therapeutic agent can be dissolved in a solvent that is compatible with both the device polymer and the therapeutic agent to form a solution. Preferably, the device polymer is at most only slightly soluble in this solvent. Subsequently, the solution is contacted with the membrane or tube such that the therapeutic agent is loaded (e.g., by leaching/diffusion) into the copolymer. For this purpose, the membrane, tube, or portions thereof can be immersed or dipped into the solution. Alternatively, the solution can be applied to the membrane, tube or portions thereof, as examples, by spraying, printing dip coating, immersing in a fluidized bed, and so forth. The loaded membrane and/or tube can subsequently be dried, with the therapeutic agent remaining therein.

In another alternative where the membrane and or tube is porous, the drug can be dissolved in a solvent and the solvent with drug vacuum impregnated into the pores of the device. The solvent can then be flashed off with or without heat with the precipitated drug remaining within the pores of the structure.

In another alternative, the therapeutic agent may be provided within a matrix comprising the polymer of the membrane or tube. The therapeutic agent can also be covalently bonded, hydrogen bonded, or electrostatically bound to the polymer of the device. As specific examples, nitric oxide releasing functional groups such as S-nitroso-thiols can be provided in connection with the polymer, or the polymer can be provided with charged functional groups to attach therapeutic groups with oppositely charged functionalities.

In yet another alternative embodiment, the therapeutic agent can be precipitated onto one or more surfaces of the membrane, tube, or portions thereof. These surfaces can be subsequently covered with a coating of polymer (with or without additional therapeutic agent) as described above.

It also may be useful to coat the polymer of the membrane or tube (which may or may not contain a therapeutic agent) with an additional polymer layer (which may or may not contain a therapeutic agent). This additional layer may serve, for example, as a boundary layer to retard diffusion of the therapeutic agent and prevent a burst phenomenon whereby much of the agent is released immediately upon exposure of the membrane or tube to the implant site. The material constituting the coating, or boundary layer, may or may not be the same polymer as the loaded polymer. For example, the barrier layer may also be a polymer or small molecule from a large class of compounds.

It is also possible to form a membrane, tube, or portions thereof for release of therapeutic agents by adding one or more of the above or other polymers to a block copolymer. Examples include the following:

a) blends can be formed with homopolymers that are miscible with one of the block copolymer phases. For example, polyphenylene oxide is miscible with the styrene blocks of polystyrene-polyisobutylene-polystyrene copolymer. This should increase the strength of a molded part or coating made from polystyrene-polyisobutylene-polystyrene copolymer and polyphenylene oxide.

b) blends can be made with added polymers or other copolymers that are not completely miscible with the blocks of the block copolymer. The added polymer or copolymer may be advantageous, for example, in that it is compatible with another therapeutic agent, or it may alter the release rate of the therapeutic agent from the block copolymer (e.g., polystyrene-polyisobutylene-polystyrene copolymer).

c) blends can be made with a component such as sugar (see list above) that can be leached from the device or device portion, rendering the device or device component more porous and controlling the release rate through the porous structure.

The release rate of therapeutic agent from the therapeutic-agent-loaded polymers of the present invention can be varied in a number of ways. Examples include but are not limited to:

a) varying the molecular weight of the block copolymers;

b) varying the specific constituents selected for the elastomeric and thermoplastic portions of the block copolymers and the relative amounts of these constituents;

c) varying the type and relative amounts of solvents used in processing the block copolymers;

d) varying the porosity of the block copolymers;

e) providing a boundary layer over the block copolymer; and

f) blending the block copolymer with other polymers or copolymers.

Moreover, although it is seemingly desirable to provide control over the release of the therapeutic agent (e.g., as a fast release (hours) or as a slow release (weeks)), it may not be necessary to control the release of the therapeutic agent.

Hence, when it is stated herein that the polymer is “loaded” with therapeutic agent, it is meant that the therapeutic agent is associated with the polymer in a fashion like those discussed above or in a related fashion.

A wide range of therapeutic agent loadings can be used in connection with the above block copolymers comprising the membrane, with the amount of loading being readily determined by those of ordinary skill in the art and ultimately depending upon the condition to be treated, the nature of the therapeutic agent itself, the means by which the therapeutic-agent-loaded copolymer is administered to the intended subject, and so forth. The loaded copolymer will frequently comprise from less than one to 70 wt % therapeutic agent.

In some instances, therapeutic agent is released from the device or device portion to a bodily tissue or bodily fluid upon contacting the same. An extended period of release (i.e., 50% release or less over a period of 24 hours) may be preferred in some cases. In other instances, for example, in the case where enzymes, cells and other agents capable of acting on a substrate are used as a therapeutic agent, the therapeutic agent may remain within the copolymer matrix.

In an alternate embodiment, the thin flexible polymeric membrane as described above can be used as an ophthalmic patch for repairing scarred, diseased or otherwise defective ocular tissue. For example, such a patch can be used in treating glaucoma such as in the repair of leaking and/or overfiltering blebs and/or repairing corneo-scleral fistulas. The patch can also be used to treat retinal disorders, such repairing exposed scleral buckles. The patch can also be used in oculoplastics, such as in eyelid reconstruction, repair of exposed orbital implants, eyelid weight cover. The patch can also be used to treat cataracts, such as to repair burns resulting from phacoemulsification. The hardness and thickness of the patch can be varied depending upon the application. In the preferred embodiment, the patch has a Shore hardness between 20 A and 90 A and a thickness between 0.020 millimeters (mm) and 0.6 mm thick (and most preferably between 0.04 mm and 0.5 mm thick). These ranges of hardness and thickness are less traumatic to the eye when the patch is implanted therein. For applications where the patch is used to prevent erosion of drainage tubes in the sclera/conjunctiva, the patch can be realized of a porous polymeric structure as described herein with a pore size in the range between 10 μm and 30 μm.

There have been described and illustrated herein several embodiments of methods and apparatus for aqueous humor drainage employing a thin flexible membrane promoting a sustained bleb as well as an ophthalmic patch realized from a thin flexible polymeric membrane. While particular embodiments of the invention have been described, it is not intended that the invention be limited thereto, as it is intended that the invention be as broad in scope as the art will allow and that the specification be read likewise. It will therefore be appreciated by those skilled in the art that yet other modifications could be made to the provided invention without deviating from its spirit and scope as claimed.

1. An apparatus for relieving pressure in an eye, comprising:
a) a tubular member having a first end opposite a second end, the first end operatively inserted into the anterior chamber of the eye, the tubular member defining a lumen providing a flowpath for drainage of aqueous humor from the anterior chamber of the eye; and
b) a flexible polymeric membrane operably disposed adjacent the second end of the tubular member outside the lumen of the tubular member, said membrane having a thickness between 0.04 mm and 0.5 mm and sized to enable growth of tissue around said membrane to form a bleb which acts as a reservoir for drainage of aqueous humor into the ocular environment.

2. The apparatus of claim 1, wherein:
said membrane is porous and allows for aqueous humor to flow therethrough.

3. The apparatus of claim 1, wherein:
said membrane is microporous.

4. The apparatus of claim 1, wherein:
said membrane is non-porous and does not allow for aqueous humor to flow therethrough.

5. The apparatus of claim 1, wherein:
at least one of said membrane and said tubular member includes polyisobutylene.

6. The apparatus of claim 1, wherein:
said membrane has a Shore hardness between 20 A and 90 A.

7. The apparatus of claim 1, wherein:
said tubular member and said membrane are integrated as a unitary piece.

8. The apparatus of claim 7, wherein:
the tubular member and membrane are integrated together by one of insert molding, suturing, melting, and solvent bonding.

9. The apparatus of claim 1, wherein:
at least one of said tubular member and said membrane is loaded with one or more drugs.

10. The apparatus of claim 9, wherein:
said one or more drugs includes one of an antiproliferative, a fibrotic agent, a anticoagulant, and a blocking agent.

11. (canceled)

12. The apparatus of claim 1, wherein:
said tubular member includes a fixation member.

13. The apparatus of claim 1, wherein:
said membrane includes a stepped interface from a smaller thickness to a larger thickness, and the second end of said tubular member is operably disposed adjacent said stepped interface.

14. The apparatus of claim 13, wherein:
the larger thickness of said stepped interface prevents the membrane from inadvertently folding.

15. The apparatus of claim 1, wherein:
said membrane covers a surface area in a range between 50 mm2 and 150 mm2.

16. A method for relieving intraocular pressure in an ocular environment, said method comprising the steps of:
a) providing a tubular member and a flexible polymeric membrane, said tubular member having a first end opposite a second end, and said membrane having a thickness between 0.04 mm and 0.5 mm;
b) inserting the first end of said tubular member into an anterior chamber of the eye, the tubular member defining a lumen providing a flowpath for drainage of aqueous humor from the anterior chamber of the eye; and
c) inserting said membrane into the ocular environment whereby the membrane is positioned adjacent the second end of the tubular member outside the lumen of the tubular member and is sized to enable growth of tissue around said membrane to form a bleb which acts as a reservoir for drainage of aqueous humor into the ocular environment.

17. The method of claim 16, wherein:
said membrane is porous and allows for aqueous humor to flow therethrough.

18. The method of claim 16, wherein:
said flexible membrane is microporous.

19. The method of claim 16, wherein:
said membrane is non-porous and does not allow for aqueous humor to flow therethrough.

20. The method of claim 16, wherein:
at least one of said membrane and said tubular member includes polyisobutylene.

21. The method of claim 16, wherein:
said membrane has a Shore hardness between 20 A and 90 A.

22. The method of claim 16, wherein:
said tubular member and said membrane are integrated together as a unitary piece.

23. The method of claim 22, wherein:
said tubular member and said membrane are integrated together by one of insert molding, suturing, melting, and solvent bonding.

24. The method of claim 16, wherein:
at least one of said tubular member and said membrane is loaded with one or more drugs.

25. The method of claim 24, wherein:
said one or more drugs includes one of an antiproliferative, a fibrotic agent, an anticoagulant, and a blocking agent.

26. (canceled)

27. The method of claim 16, wherein:
said tubular member includes a fixation member.

28. The method of claim 16, wherein:
said membrane includes a stepped interface from a smaller thickness to a larger thickness, and the second end of said tubular member is operably disposed adjacent said stepped interface.

29. The method of claim 28, wherein:
the larger thickness of said stepped interface prevents the membrane from inadvertently folding.

30. The method of claim 16, wherein:
said membrane covers a surface area in a range between 50 mm2 and 150 mm2.

31. The method of claim 16, wherein:
said membrane is positioned under the conjunctiva of the eye.

32. The method of claim 31, wherein:
said membrane is positioned into a flap in the eye formed between the conjunctiva/Tenon's connective tissues and the sclera.

33. An apparatus for treating a disorder of the eye, comprising:
a flexible membrane realized from a block copolymer including polyisobutylene, wherein said flexible membrane has a thickness between 0.04 mm and 0.5 mm.

34. The apparatus of claim 33, wherein:
one or more drugs are loaded into said block copolymer.

35. The apparatus of claim 34, wherein:
said one or more drugs treats macular degeneration.

36. The apparatus of claim 34, wherein:
said one or more drugs treats glaucoma.

37. The apparatus of claim 34, wherein:
said one or more drugs includes one of an antiproliferative, a fibrotic agent, an anticoagulant, and a blocking agent.

38. The apparatus of claim 34, wherein:
said flexible membrane has a Shore hardness between 20 A and 90 A.

39. The apparatus of claim 34, wherein:
said flexible membrane is porous.

40. The apparatus of claim 34, wherein:
said flexible membrane is microporous.

41. The apparatus of claim 34, wherein:
said flexible membrane is non-porous.

42. An apparatus for relieving pressure in an eye, comprising:
a) a tubular member having a first end opposite a second end, the first end operatively inserted into the anterior chamber of the eye, the tubular member defining a lumen providing a flowpath for drainage of aqueous humor from the anterior chamber of the eye; and
b) a flexible polymeric membrane operably disposed adjacent the second end of the tubular member outside the lumen of the tubular member and sized to enable growth of tissue around said membrane to form a bleb which acts as a reservoir for drainage of aqueous humor into the ocular environment;
wherein said membrane has a thickness between 0.04 mm and 0.5 mm and has a Shore hardness between 20 A and 90 A, and wherein at least one of said membrane and said tubular member includes polyisobutylene.

43. A surgical device for repairing ocular tissue comprising:
a flexible membrane of a polymeric material comprising polyisobutylene.

44. The surgical device of claim 43, wherein:
said polymeric material is porous.

45. The surgical device of claim 43, wherein:
said flexible membrane has a thickness between 0.04 mm and 0.5 mm.

46. The surgical device of claim 43, wherein:
said flexible membrane has a Shore hardness between 20 A and 90 A.
